(19) United States
(12) Patent Application Publication
(10) Pub. No.: US 2012/0316538 A1
(43) Pub. Date: Dec. 13, 2012

(54) Osmotic Wound Vacuum System

(76) Inventors: Jeremy Heiser, Salt Lake City, UT (US); Ashok V. Joshi, Salt Lake City, UT (US); John Howard Gordon, Salt Lake City, UT (US)

(21) Appl. No.: 13/592,081

(22) Filed: Aug. 22, 2012

Related U.S. Application Data
(63) Continuation-in-part of application No. 13/189,107, filed on Jul. 22, 2011.
Application No. 13/189,107 is a division of application No. 11/958,303, filed on Dec. 17, 2007, now Pat. No. 8,012,169.
Application No. 11/958,303 is a division of application No. 10/657,820, filed on Sep. 8, 2003, now Pat. No. 7,361,184.
(60) Provisional application No. 61/526,187, filed on Aug. 22, 2011.

(51) Int. Cl. A61M 27/00

(52) U.S. Cl. 604/543

(57) ABSTRACT

A negative pressure wound therapy (NPWT) system creates and maintains a sub-atmospheric pressure within a sealed wound environment for the purpose of healing wounds. The NPWT system includes a wound interface material, a housing, an osmotic membrane, and an evacuation port. The wound interface material contacts a wound region and absorbs exudate from the wound region. The housing defines a cavity in an interior space of the housing. The osmotic membrane is coupled to the wound interface material to transfer a fluid of the exudate from the wound interface material to the cavity of the housing. The evacuation port is also coupled to the wound interface material to facilitate passage of a gas out of the wound interface material to create a negative pressure at the wound region.

### CROSS-REFERENCE TO RELATED APPLICATIONS

This application is a continuation-in-part of U.S. patent application Ser. No. 13/189,107 filed Jul. 22, 2011 and entitled “Electrochemical Wound Therapy Device,” which is a divisional of U.S. patent application Ser. No. 11/958,303, filed on Dec. 17, 2007, now U.S. Pat. No. 8,012,169, which is a divisional of U.S. patent application Ser. No. 10/657,820, filed on Sep. 8, 2003, now U.S. Pat. No. 7,361,184. This application also claims the benefit of U.S. Provisional Application No. 61/526,187, filed on Aug. 22, 2011. The disclosure of each of these applications is incorporated herein by reference.

### BACKGROUND

The concept of using negative pressure wound therapy (NPWT) to promote the healing of open wounds has been shown to result in faster wound healing than other conventional methods. Unfortunately, conventional NPWT systems are typically bulky, complicated, and expensive. The high cost of treating patients with the conventional NPWT systems may be a factor in dramatically limiting the use of NPWT systems in low-resource settings such as developing countries.

### SUMMARY

Embodiments of a negative pressure wound therapy (NPWT) system are described. In an embodiment, the NPWT system creates and maintains a sub-atmospheric pressure within a sealed wound environment for the purpose of healing wounds. The NPWT system may include a wound interface material, a housing, an osmotic membrane, and an evacuation port. The wound interface material contacts a wound region and absorbs exudate from the wound region. The housing defines a cavity in an interior space of the housing. The osmotic membrane is coupled to the wound interface material to transfer a fluid of the exudate from the wound interface material to the cavity of the housing. The evacuation port is also coupled to the wound interface material to facilitate passage of a gas out of the wound interface material to create a negative pressure at the wound region.

In another embodiment, the NPWT system includes the wound interface material, the osmotic membrane, and a pump. The wound interface material contacts a wound region and absorbs exudate from the wound region. The osmotic membrane transfers a fluid of the exudate out of the wound interface material. The pump is coupled to the wound interface material via an evacuation port. The pump is in fluid communication with the wound interface material to pump a gas out of the wound interface material to create a negative pressure at the wound region.

In another embodiment, the NPWT system includes the housing, the osmotic membrane, the wound interface material, and the pump. The housing defines a cavity in an interior space of the housing. The osmotic membrane is disposed at an opening to the cavity of the housing. The wound interface material is in direct physical contact with at least a portion of the osmotic membrane to absorb exudate from a wound region. The osmotic membrane transfers a fluid of the exudate from the wound interface material to the cavity of the housing. The pump at least partially evacuates a gas out of the wound interface material to create a negative pressure at the wound region. Other embodiments of the system are also described.

Other aspects and advantages of embodiments of the present invention will become apparent from the following detailed description, taken in conjunction with the accompanying drawings, illustrated by way of example of the principles of the invention.

### BRIEF DESCRIPTION OF THE DRAWINGS

FIG. 1 illustrates a cross-sectional view of one embodiment of a negative pressure wound therapy (NPWT) system that may sit flush on a wound region.

FIG. 2 illustrates a cross-sectional view of another embodiment of a NPWT system with a wound interface material which extends beyond the bottom face of the housing.

FIG. 3 illustrates a cross-sectional view of another embodiment of a NPWT system with a wound interface material which extends beyond the lateral bounds of the housing.

FIG. 4 illustrates a cross-sectional view of another embodiment of a NPWT system with a remotely located pump.

FIG. 5 illustrates a view of one embodiment of the multi-valve attachment of FIG. 4.

FIG. 6A illustrates a view of another embodiment of a multi-valve attachment compatible with the system of FIG. 4 to provide visual feedback of pressure.

FIG. 6B illustrates another view of the multi-valve attachment of FIG. 6A after movement of the check valve relative to the measurement markings.

FIG. 7 illustrates a cross-sectional view of another embodiment of a NPWT system with a detachable pump.

FIG. 8 illustrates a cross-sectional view of another embodiment of a NPWT system with a remote housing.

FIG. 9 illustrates a cross-sectional view of another embodiment of a NPWT system with a pump that is located at the wound interface material separately from the remotely located housing.

FIG. 10 illustrates a cross-sectional view of another embodiment of a NPWT system with a remote housing and a remote pump.

FIG. 11 illustrates a cross-sectional view of another embodiment of a NPWT system with a remote housing and a detachable pump.

FIG. 12 illustrates a cross-sectional view of another embodiment of a NPWT system with a pump located within the housing.

FIG. 13 illustrates a cross-sectional view of another embodiment of a NPWT system with a pump located within a remote housing.

FIG. 14 illustrates a cross-sectional view of another embodiment of a NPWT system with a pump located within a remote housing and a gas channel extending from the pump to the dressing portion of the wound interface material.

FIG. 15 illustrates a cross-sectional view of another embodiment of a NPWT system with an expandable reservoir which also forms the outer housing.

FIG. 16 illustrates a schematic diagram of one embodiment of a NPWT system with a feedback system.

Throughout the description, similar reference numbers may be used to identify similar elements.

### DETAILED DESCRIPTION

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the indicated embodiment is included in at least one embodiment of the present invention. Thus, the phrases “in one embodiment,” “in an embodiment,” and similar language throughout this specification may, but do not necessarily, all refer to the same embodiment.

While many embodiments are described herein, at least some of the embodiments include a negative pressure wound therapy (NPWT) system that creates and maintains a sub-atmospheric pressure within a sealed wound environment to promote healing wounds. Embodiments of the system are capable of producing a negative pressure over the wound region through a combination of osmotic removal of the fluid portion of the wound exudate and mechanical removal of gases within the wound environment. Embodiments of the NPWT system offer a low cost, disposable wound-healing device that provides a simplified method to administer NPWT to individuals in low resource settings. In some embodiments, the NPWT system is a single-use, disposable system.

Some embodiments described herein create a new product category which includes the use of a secondary pump for the removal of gases from the wound environment and the osmotic transfer of the fluid portion of wound exudate from the wound environment to a secondary reservoir for the purpose of creating a vacuum within the wound environment. This category is different from the existing product categories in that the vacuum source is a combination of mechanical gas removal and osmotic fluid removal and that wound exudate is not removed from the wound environment, nor is it completely contained within the wound environment. Thus, some embodiments described herein remove only the fluid portion of the exudate from the wound environment, leaving the cellular material and debris within the dressing.

Embodiments of the NPWT system may have advantages over conventional NPWT systems. Some of these advantages of specific embodiments may include, but are not necessarily limited to: relatively low cost because there is no need for a battery pack and controls; relatively low weight which facilitates a ambulatory solution that is less cumbersome to wear during daily activities; relatively small size making it easier to use with less impact on user mobility; relatively low maintenance because there may be no need to replace secondary canisters or removable exudate reservoirs; relatively low-power or no-power because by using a mechanical hand pump or other low-power or no-power pump; and relatively quiet operation which limits the amount of interruption and/or disturbance to a patient's other activities. Other embodiments may exhibit other advantages over conventional systems.

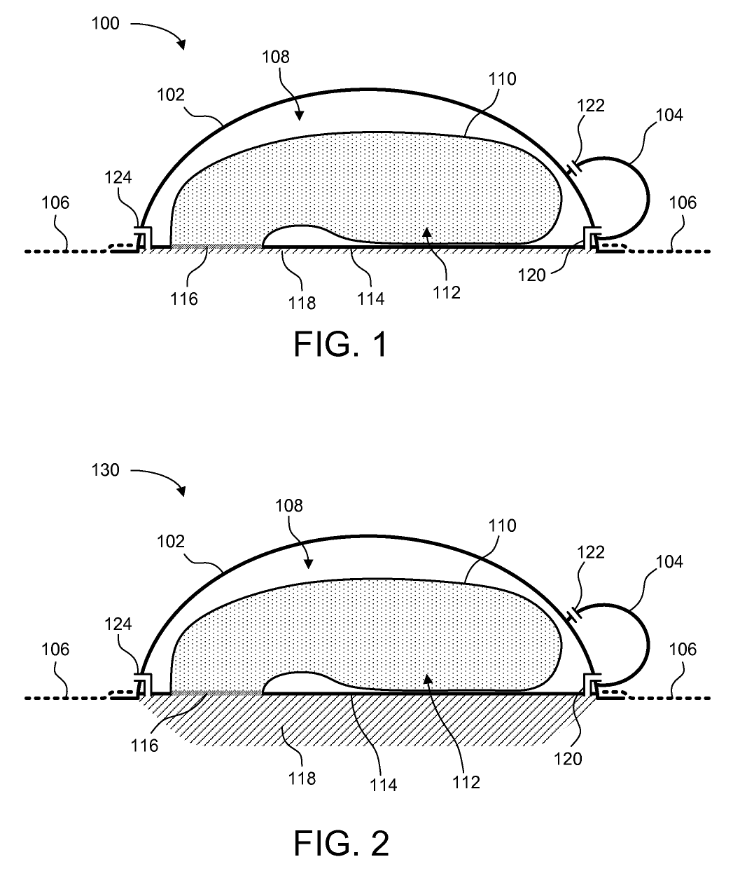

FIG. 1 illustrates a cross-sectional view of one embodiment of a negative pressure wound therapy (NPWT) system 100 that may sit flush on a wound region. In general, the NPWT system 100 functions to cover and protect a wound region, as well as create negative pressure at the wound region to promote healing. Although the NPWT system 100 is shown and described with certain components and functionality other embodiments may include fewer or more components to implement the same, similar, or additional functionality.

The illustrated NPWT system 100 includes a housing 102, a pump 104, a sealing member 106, a cavity 108, an expandable reservoir 110, an osmogent material 112, a bottom panel 114 with an opening, an osmotic membrane 116, and a wound interface material 118. The NPWT system 100 also includes an evacuation port 120, a discharge port 122, and a relief port 124.

The housing 102 may be fabricated of any suitable material including, but not limited to, a rigid material, a semi-rigid material, a flexible material, or a combination of materials. The use of a rigid or semi-rigid material for the housing 102 may provide some structural stability and/or protection for fluids or other components or materials within the housing 102. In some embodiments, the housing 102 defines a single cavity 108 or compartment within the interior space of the housing 102. In other embodiments, the housing 102 may define two or more internal cavities or compartments. Each cavity or compartment may be used to house one or more components or materials.

Also, the housing 102 may be made of up of one or more individual pieces. The individual pieces may be arranged and coupled together in a fixed manner. Alternatively, some or all of the individual pieces may be arranged and coupled together to allow for movement between adjacent pieces. This arrangement may allow for contraction or expansion of the cavity 108 within the housing 102.

Additionally, the housing may be fabricated or arranged in any suitable shape and size. In the illustrated embodiments, the housing 102 has a shape that somewhat resembles a spheroidal dome. In other embodiments, the housing 102 may be fabricated or arranged in the shape of a cylinder, sphere, cube, box, or any other shape. In some embodiments, the shape of the housing 102 may be designed to conform to a corresponding body surface of a patient. For example, the shape of the housing may be ergonomically influenced to conform to an appendage (e.g., arm, leg, etc.) or region (e.g., head, thorax, etc.) of a human body. Any form of securing the housing 102 to a patient may be used including, but not limited to, adhesive, a strap, a wrap, a clip, or a combination of these or other securing means.

In one embodiment, the housing 102 includes a bottom panel 114 or surface. For convenience, as used herein the bottom surface is a surface that is anticipated to make contact with or be disposed faced a wound region of a patient. However, other orientations and directional conventions may be used. In some embodiments, the bottom panel 114 has a curved surface to accommodate an anatomical feature at a wound region or surrounding a wound region. In some embodiments, the bottom panel 114 or surrounding portions of the housing 102 may be pliable to conform to the region surrounding a wound.

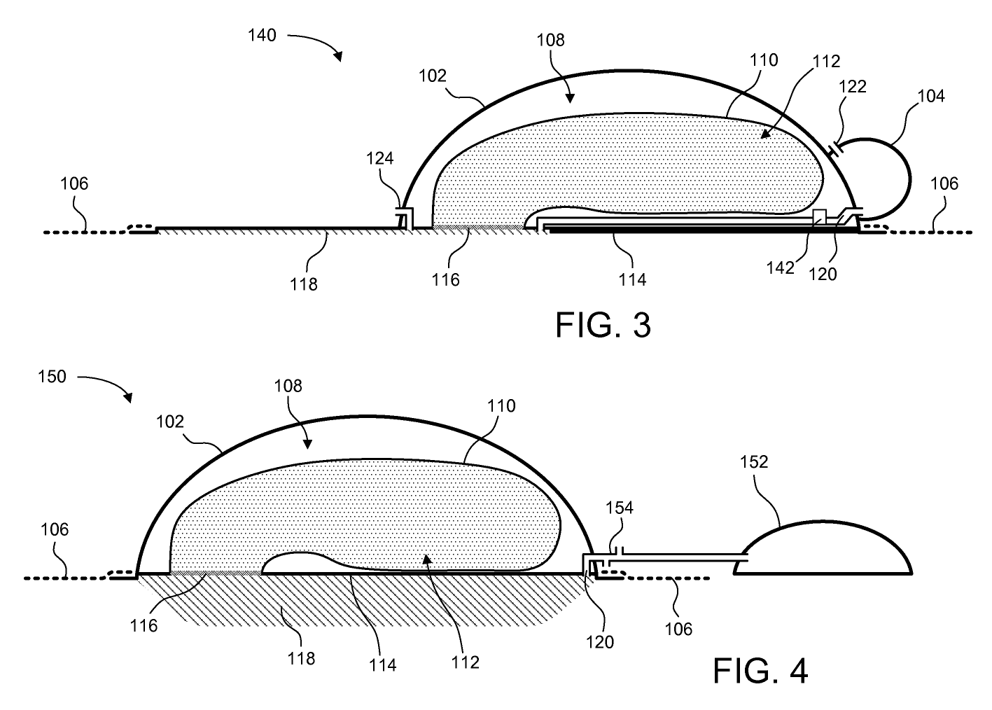

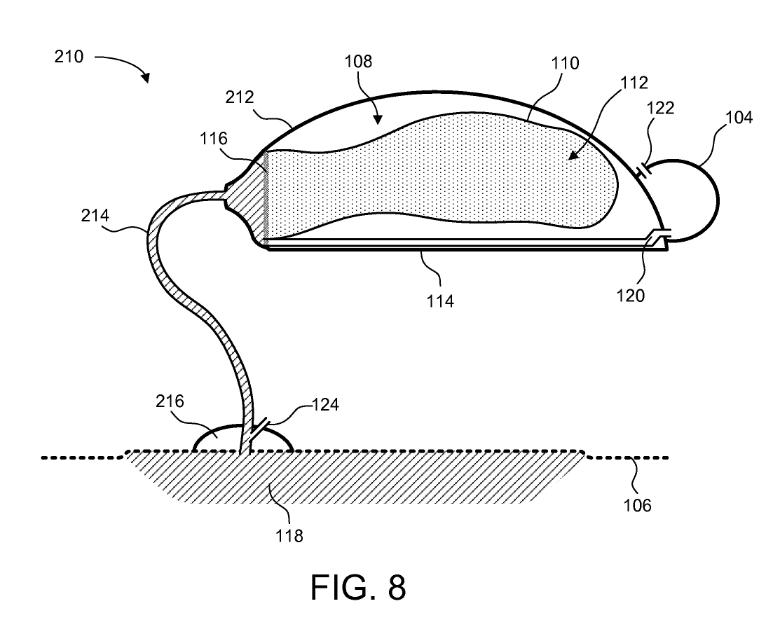

In the depicted embodiment, the wound interface material 118 is attached to or otherwise disposed at the bottom panel 114 of the housing. In some embodiments, the wound interface material 118 may cover essentially the entire bottom panel 114 of the housing 102. Alternatively, the wound interface material 118 may cover only a portion of the bottom surface (see FIG. 3) or may not be in contact with an outer surface of the housing (see FIG. 8).

The wound interface material 118 may include any material (or combination of materials) that facilitates storage and/or transport of fluids at the wound region. In some embodiments, the wound interface material 118 is a wicking material, an absorptive material, a hydrophilic material, or another type of material. In further embodiments, the wound interface material 118 also includes an antimicrobial agent and/or a medicinal agent for application at the wound region. Also, in some embodiments, the wound interface material 118 includes one or more layers of materials. The layers of materials may include a wound contact layer, and exudate transfer layer, an exudate control layer, a cellular material management layer, a wound protein management layer, an exudate solids management layer, a bio-burden control layer, an odor management layer, a wound environment monitoring layer, and/or a growth factor delivery layer.

For example, a wound contact layer may include without limitation: Profore, a Smith and Nephew product; DelNet, a DelStar product; Tegapore from 3M; Silon-TSR® Temporary Skin Replacement by Bio Med Sciences; DERMANET by DeRoyal; TELFA CLEAR by Kendall; Mepitel by Molnlycke Health Care; N-TERFACE by Winfield Laboratories; Medifil Skin Temp by BioCore; BGC Matrix by Brennen; WOUN'DRESS by Coloplast Sween; Collagen/AG by DermaRite; ColActive Ag by Hartmann-Conco, Inc.; FIBRACOL plus Collagen Prisma Promogran Prisma by Johnson & Johnson; Biostep Biostep Ag by Smith & Nephew; Stimulen by Southwest; Primatrix by TEI Biosciences; and/or CellerateRx by Wound Care Innovations. A exudate transfer layer may include by way of nonlimiting example: Coloplast by Milliken; and/or Profore by Smith and Nephew. An exudate control layer may include: Medipore by 3M; Silon Dual-Dress 04P® Multi-Function Wound Dressing & Silon Dual-Dress 20F® Multi-Function Wound Dressing by Bio Med Sciences; Aquacel Hydrofiber CombiDERM by ConvaTec; Absorptive Border by DermaRite; MULTIPAD SOFSORB by DeRoyal; IODOFLEX by HEALTHPOINT; TIELLE by Johnson & Johnson; CURITY ABD and TELFAMAX TENDERSORB ABD by Kendall; Mepore by Molnlycke Health Care; and/or EXU-DRY Primapore by Smith & Nephew. The exucdate control layer may include other alginates or hydrolloids known in the art.

The wound interface material 118 may include a bio-burden control layer and may be impregnated with Silver. Examples of a bio-burden control layer may include the following products: 3M Tegaderm Ag Mesh by 3M; SilverDerm7 by DermaRite; SelectSilver by Milliken Company; or other products known in the industry. An odor management layer may include activated charcoal, and may include products such as CarboFlex or LyoFoam C by ConvaTec. A wound environment monitoring layer, a transparent layer for visual inspection, a moisture level monitoring layer and/or a growth factor delivery layer may include the following products: Apligraf by Organogenesis; Dermagraft Transcyte by Advanced Biohealing; Orcel by Ortec International, Inc.; or other products known in the art. A cellular material management layer, a wound protein management layer, and or an exudate solids management layer may include non-woven material, gause, foam, or other materials. These materials may or may not be impregnated with other materials.

In the illustrated embodiment, the wound interface material 118 is mounted to, or in contact with, the bottom panel 114 of the housing 102 so that the exposed side of the wound interface material 118 is substantially flush with the bottom perimeter edges, or contour, of the housing 102. In some embodiments, the housing 102 includes one or more lateral flanges that extend outward from the body of the housing 102. In these embodiments, the wound interface material 118 may be disposed so as to be flush with the bottom surface of the flanges. In other embodiments, some or all of the exposed wound interface material may extend outward (vertically in FIG. 1) from the bottom panel 114 of the housing 102.

In addition to defining a bottom contour of the housing 102, the lateral flanges may provide a contact surface for one or more sealing member 106. In general, the sealing member 106 functions to create a sealed wound environment at the wound region. The sealed wound environment is a substantially contained environment in which essentially all gas and fluid flow into or out of the sealed wound environment is prevented, with the exception of fluid flow into the housing, as described below. In this way, a negative pressure can be created and maintained (at least for a measurable and useful time) within the sealed wound environment to promote healing at the wound region. The sealing member 106 by itself may be in direct physical contact with some or all of the wound interface material 118 and cover the entire wound region. Alternatively, the sealing member 106 may be in direct physical contact and function with some or all of the housing 102 to cover the entire wound region. In either case, the sealing member 106 may be any material with adhesive properties and a relatively high level of impermeability to gases and fluids. Some examples of such materials include, but are not limited to, an occlusive or semi-occlusive film adhesive.

In the illustrated embodiment, the bottom panel 114 of the housing 102 defines an opening. An osmotic membrane 116 is disposed at the opening. In this embodiment, the osmotic membrane 116 and the wound interface material 118 are in contact with each other at approximately the bottom panel 114 of the housing 102. The wound interface 118 transfers the wound exudate from the wound region to the corresponding surface of the osmotic membrane 116. The osmotic membrane 116 is also in contact with the osmogent material 112 within the expandable reservoir 110 inside the cavity 108 of the housing 102. In some embodiments, the osmogent material 112 creates an osmotic gradient which draws a fluid component of the exudate at the wound region through the osmotic membrane 116 and into the expandable reservoir 110. Drawing fluid out of the wound region, within the sealed wound environment, contributes to the negative pressure within the sealed wound environment. In some embodiments, the housing 102 protects the expandable reservoir 110 from compression.

The illustrated NPWT system 100 also includes a pump 104. The pump 104 is coupled to an exterior surface of the housing 102. However, the pump 104 may be connected to the wound interface material 118 through other means. In general, the pump 104 evacuates gas from the sealed wound environment to further contribute to the negative pressure within the sealed wound environment. The pump 104 may be any type of pump. For example, the pump 104 may be an electromechanical pump or a manually powered hand pump. As other examples, the pump 104 may be a reciprocating pump, a gear-driven pump, a crank-driven pump, a progressing cavity pump, peristaltic pump, or a diaphragm pump.

The pump is coupled to and in fluid communication with an evacuation port 120. Through the evacuation port 120, the pump is in fluid communication with the wound interface material 118 so that the pump 104 can evacuate gases and/or fluids out of the wound interface material 118.

The pump is also coupled to a gas discharge port 122. The gas discharge port 122 allows for the expulsion of gases from the pump 104 into the ambient environment. This, in turn, allows the gas evacuation port 120 to remove gas from the wound region.

In some embodiments, a pressure relief port 124 allows for a controlled inlet of gas from the ambient environment into the sealed wound environment for the purpose of maintaining a therapeutic vacuum level within the sealed wound environment and protecting the wound. For example, if the vacuum pressure within the sealed wound environment goes too far negative (e.g., below a threshold), then the relief port 124 may allow an amount of gas to enter the sealed wound environment to rebalance the negative pressure at a predetermined or desired level. This allows any level of vacuum pressure to be maintained within the sealed wound environment. For example, the relief port 124 may maintain the negative pressure within the sealed wound environment in a range of about 25-500 mmHg. As another example, the relief port 124 may maintain the negative pressure within the sealed wound environment at about 125 mmHg.

FIG. 2 illustrates a cross-sectional view of another embodiment of a NPWT system 130 with a wound interface material 118 which extends beyond the bottom face of the housing 102. More specifically, the wound interface material 118 is relatively thick so that the wound interface material 118 extends below the outer edge, or contour, of the housing 102. Thus, while a top surface of the wound interface material 118 is flush with the bottom panel 114 or surface of the housing 102, and a bottom surface of the wound interface material 118 extends outward from the bottom panel 114 of the housing 102. The use of a thicker wound interface material 118 may be useful in situations where additional fluids are present or where the wound region is relatively deep.

FIG. 3 illustrates a cross-sectional view of another embodiment of a NPWT system 140 with a wound interface material 118 which extends beyond the lateral bounds of the housing 102. In this way, the wound interface material 118 is at least partially offset from the housing 102, although it is still aligned or in contact with the osmotic membrane 116. Offsetting the housing 102 in this manner may provide some additional accessibility to the wound region, without significantly disturbing the placement of the housing 102 and the pump 104.

The extension of the wound interface material 118 beyond the boundary of the housing 102 may be covered by any suitable material. In one example, a flange may extend from the body of the housing 102. In another example, a sealing member 106 may be used to cover the offset portion of the wound interface material 118.

In some embodiments, depending on the location of the pump 104 relative to the wound region, the NPWT system 100 may include one or more tubes, channels, or lumens, between the wound interface material 118 and the pump 104. Also, in some embodiments, a filter 142 is coupled in fluid communication with the evacuation port 120. The filter 142 may restrict transfer of fluid and/or solid components of the exudate through the evacuation port 120 so that only gases pass through to the pump 104. The filter 142 may include an antibacterial material to restrict transfer of bacteria from the wound region to the pump 104 or to the ambient environment outside of the wound interface material 118.

FIG. 4 illustrates a cross-sectional view of another embodiment of a NPWT system 150 with a remotely located pump 152. The pump 152 is similar to the pump 104 described above, except that it is coupled to the housing 102 by a tube or channel and a valve 154. In some embodiments, the valve 154 includes both the evacuation port and the relief port. In other embodiments, the valve 154 may include one or more ports of the different types of ports described herein. By remotely locating the pump 152 away from the housing 102, patients who use the NPWT system 100 may find convenience and, or ease of use by placing or storing the pump in a quieter or more travel-friendly location.

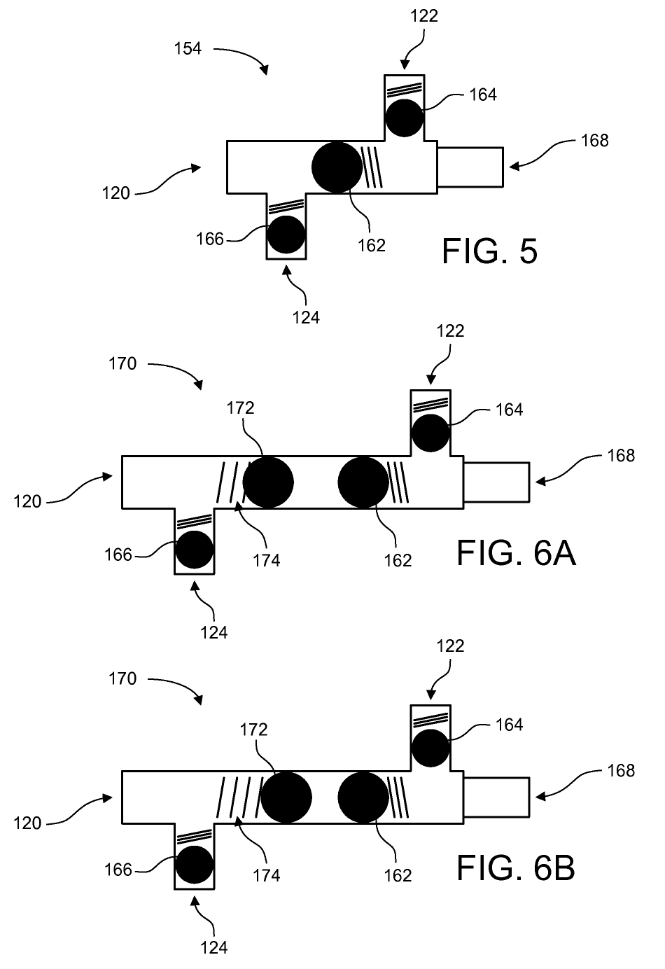

FIG. 5 illustrates a view of one embodiment of the multi-valve attachment 154 of FIG. 4. In the illustrated embodiment, the multi-valve attachment 154 is a pressure control system with a three-way valve arrangement. The three-way valve arrangement may be coupled to the gas tube or channel between the pump 104 and the evacuation port 120. In some embodiments, the three-way valve arrangement includes the evacuation port 120 (or a connection compatible with the evacuation port), the discharge port 122 to discharge the gas evacuated out of the wound interface material into an ambient space, and the 124 relief port to maintain the negative pressure within the sealed wound environment above a threshold.

In some embodiments, the elements of the multi-valve system 154 function to control and regulate the level of negative pressure in the sealed wound environment through a combination of check valves. Gas may be drawn by the pump 104 from the gas evacuation port 120 through a wound environment check valve 162 and into the vacuum path 168. In the event that the pump 104 is not drawing gas from the sealed wound environment, the wound environment check valve 164 closes and effectively maintains the wound environment seal. In the event that excessive negative pressure is produced in the sealed wound environment, the pressure relief check valve 166 at the pressure relief port 124 opens and allows the controlled introduction of ambient gas into the sealed wound environment, thereby reducing the excessive negative pressure to a predetermined or desired level. In the event that the pump 104 expels gas as part of its operation, for example a compressible bulb, the discharge check valve 164 allows for the expulsion of gas through the gas discharge port 122 into the ambient surroundings. When the pump 104 is drawing gas from the wound environment, the discharge check valve 164 seals the gas discharge port 122 allowing gas to be drawn through the multi-valve component 154 and into the vacuum path 168.

FIG. 6A illustrates a view of another embodiment of a multi-valve attachment 170 compatible with the NPWT system 150 of FIG. 4. The multi-valve attachment 170 facilitates visual feedback of pressure. In addition to the components shown in FIG. 5, the multi-valve attachment 170 of FIG. 6A also includes an indicator check valve 172 and a feedback window 174. In general, the illustrated feedback system functions to provide feedback regarding the vacuum level within the sealed wound environment. More specifically, in one embodiment a position of the pressure indicator check valve 172 relative to one or more measurement markings (e.g., printed lines, formed ridges, etc.) of the feedback window 174 is indicative of a level of the negative pressure within the wound interface material 118. In one embodiment, the pressure indicator check valve 172 (e.g., a ball) moves into the feedback window 174 when the vacuum level of the sealed wound environment is within a specified range or at a specified level, as shown in FIG. 6A, thereby providing feedback that the vacuum level within the sealed wound environment is appropriate.

FIG. 6B illustrates another view of the multi-valve attachment 170 of FIG. 6A after movement of the check valve 172 relative to the measurement markings of the feedback window 174. In particular, the pressure indicator check valve 172 is outside of the measurement markings of the feedback window 174, thereby providing feedback that the vacuum level within the sealed wound environment is not appropriate.

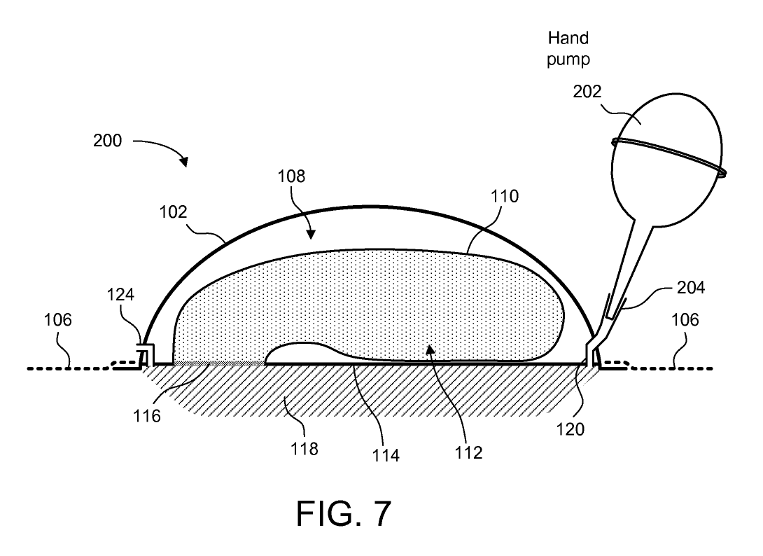

FIG. 7 illustrates a cross-sectional view of another embodiment of a NPWT system 200 with a detachable pump 202. By implementing an embodiment with a detachable pump 202, it may be possible for a patient to continually wear the housing 102 without continually wearing the pump 202. This may make it more convenient for the patient, as the housing 102 may be relatively small compared with the size and weight of the pump 202. In the depicted embodiment, the detachable pump 202 is a bulb pump that expels air through a nozzle when the bulb is compressed and draws air into the bulb when the bulb elastically expands (when external pressure is removed from the bulb structure).

The depicted NPWT system 200 also includes a pump interface 204 at which the nozzle (or other attachment) of the detachable pump 202 can be connected. In one embodiment, the pump interface 204 is an opening into which the nozzle of the detachable pump 202 can be inserted. The size and shape of the opening may vary, depending on the type of connection used for the detachable pump 202. In some embodiments, insertion of the pump attachment into the pump interface 202 (or otherwise connecting the detachable pump 202 to the pump interface 204) forms a seal between the detachable pump 202 and the pump interface 204.

FIG. 8 illustrates a cross-sectional view another embodiment of a NPWT system 210 with a remote housing 212. In this embodiment, the housing 212, the osmotic components 116 and 112, and the pump 104 are located remotely from the wound region. Also, the bottom panel 114 of the housing 212 does not function as an upper surface of the sealed wound environment. Rather, the sealing member 106 extends over substantially all of the wound interface material 118 to create and maintain a sealed wound environment. A tube or channel 214 connects the remote housing 212 to a dressing attachment 216 at the wound region. The tube 214 and dressing attachment 216 may be any form of flexible, semi-flexible, or rigid material. Also, in the illustrated embodiment, the pressure relief port 124 is located at the dressing attachment 216. However, in other embodiments, the pressure relief port 124 may be located at another location within the NPWT system 210.

In this embodiment, the wound interface material 118 includes two portions. A dressing portion is located at the wound region, and a tube portion is contained in the tube 214 which connects the dressing portion to the osmotic membrane 216. The dressing portion of the wound interface material 118 transfers the exudates fluid from the wound region to the dressing attachment 216, and the tube portion of the wound interface material 118 transfers the fluid from the dressing portion of the wound interface material 118 to the osmotic membrane 116 at the remote housing 212. The dressing portion and the tube portion of the wound interface material 118 also transfer gases through the dressing attachment 216 and the tube 214 to facilitate evacuation of gases through the evacuation port 120.

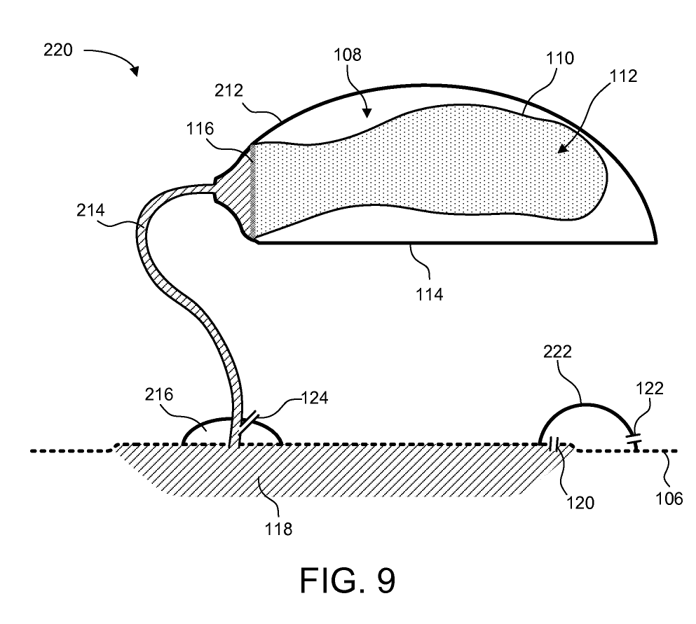

FIG. 9 illustrates a cross-sectional view of another embodiment of a NPWT system 220 with a pump 222 that is located at the wound interface material 118 separately from the remotely located housing 212. As shown in FIG. 8, the remote housing 212 and the osmotic components 116 and 112 are located adjacent to, but separate from, the dressing portion of the wound interface material 118. The remote housing 212 is to the dressing portion of the wound interface material 118 via the tube or channel 214. In this embodiment, the pump 222 is coupled to and/or integrated with the dressing portion of the wound interface material 118 and/or the sealing member 106.

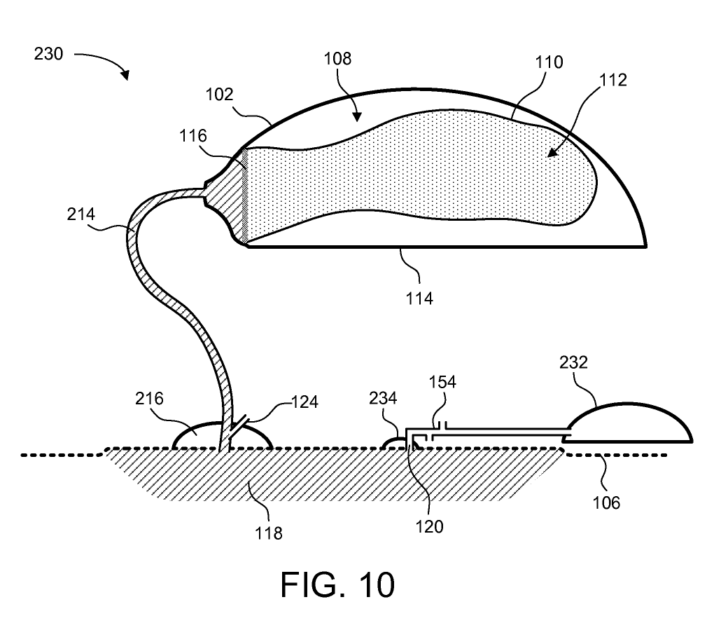

FIG. 10 illustrates a cross-sectional view of another embodiment of a NPWT system 230 with a remote housing 212 and a remote pump 232. The remote pump 232 is attached to the dressing portion of the wound interface material 118 by a pump attachment 234 and a multi-valve attachment 154. Although the pressure relief port 124 is shown located at the dressing attachment 216, in some embodiments the pressure relief port 124 may be located at the pump attachment 234.

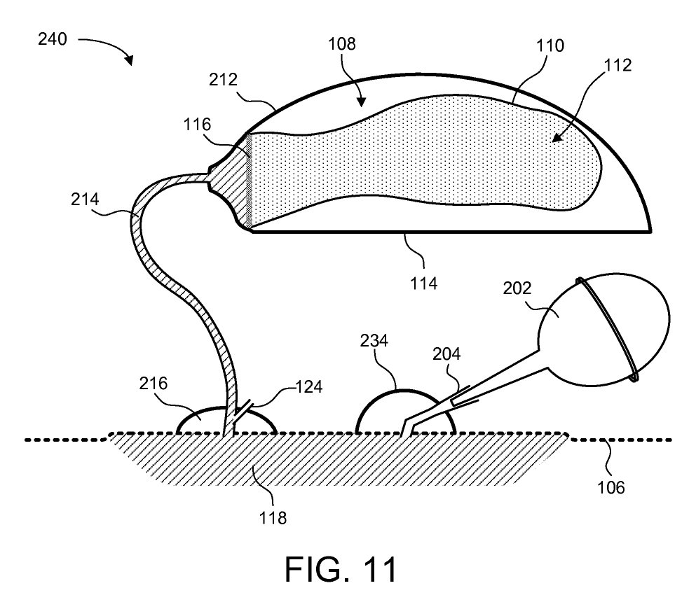

FIG. 11 illustrates a cross-sectional view of another embodiment of a NPWT system 240 with a remote housing 212 and a detachable pump 202. The detachable pump 202 can be connected to the dressing portion of the wound interface material 118 via a pump interface 204 at the pump attachment 234.

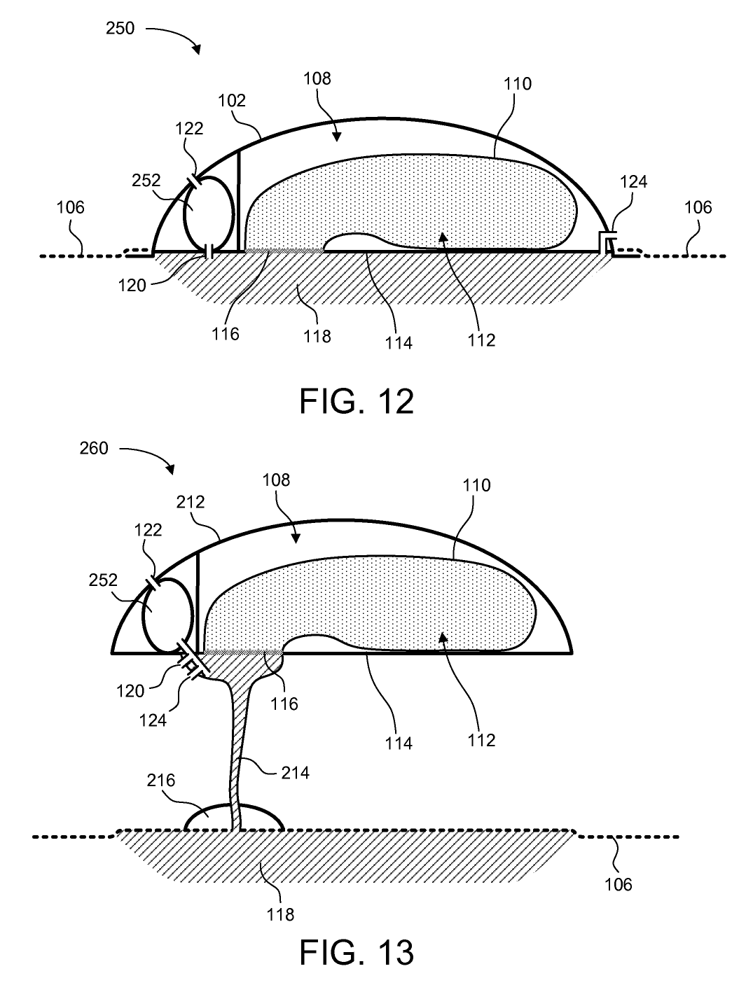

FIG. 12 illustrates a cross-sectional view another embodiment of a NPWT system 250 with a pump 252 located within the housing 102. In some embodiments, the housing 102 defines multiple interior compartments or cavities. In one of those cavities, a pump 252 may be integrated within the housing 102. Alternatively, the pump 252 may be contained in the same cavity 108 as the expandable reservoir 110.

In the illustrated embodiments, the evacuation port 120 passes directly through the bottom panel 114 of the housing 102, and the gas discharge port 122 passes through an upper structure of the housing 102.

FIG. 13 illustrates a cross-sectional view of another embodiment of a NPWT system 260 with a pump 252 located within a remote housing 212. The remote housing 212 is coupled to the wound region by a tube or channel 214 that contains a tube portion of the wound interface material 118. In this embodiment, the pump 252 is attached to one or more gas channels which extend into the tube portion of the wound interface material 118. The gas channels within the tube 214 include the evacuation port 120 and the pressure relief port 124.

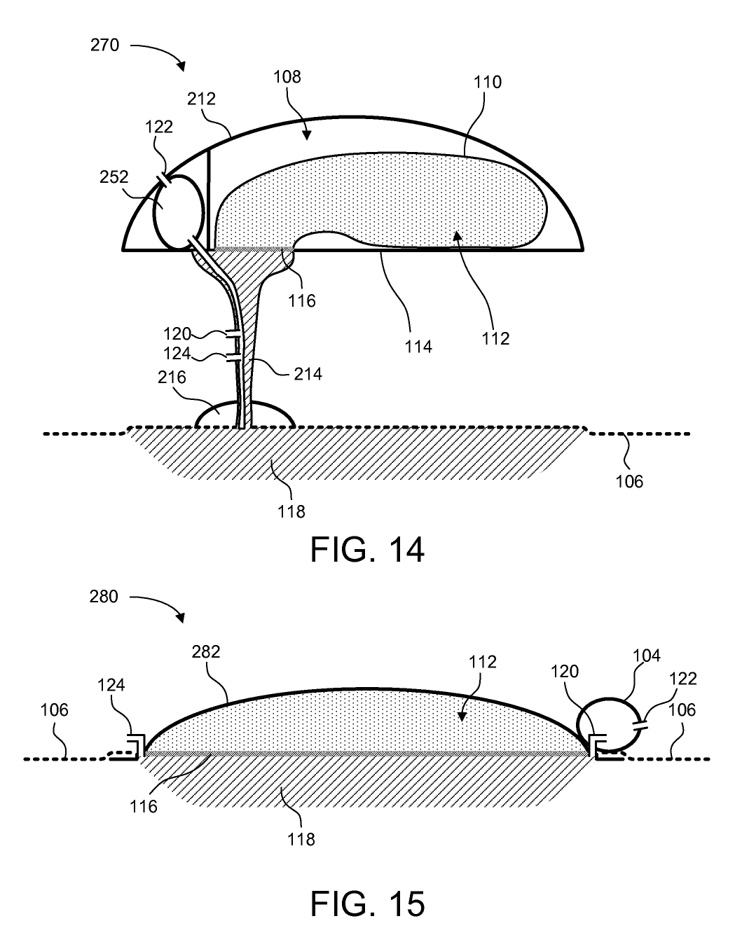

FIG. 14 illustrates a cross-sectional view of another embodiment of a NPWT system 270 with a pump 252 located within a remote housing 212 and a gas channel extending from the pump 252 to the dressing portion of the wound interface material 118. The gas channel that extends from the pump 252 to the dressing portion of the wound interface material 118 may include one or more lumens within the same tube or in separate tubes. Similar to the embodiment shown in FIG. 13, the evacuation port 120 and the gas discharge port 122 extend directly out of the tube portion of the wound interface material 118.

FIG. 15 illustrates a cross-sectional view of another embodiment of a NPWT system 280 with an expandable reservoir 282 which also forms the outer housing. In other words, the expandable reservoir 282 is not enclosed in a separate housing component. In some embodiments, the expandable reservoir 282 is made of a resiliently durable material that is both expandable and, yet, provides some structural protection for the wound region below. Also, in some embodiments, the osmotic membrane 116 may extend across substantially the entire bottom interface of the expandable housing 282. In this way, the osmotic membrane 116 may cover the entire span of the wound interface material 118.

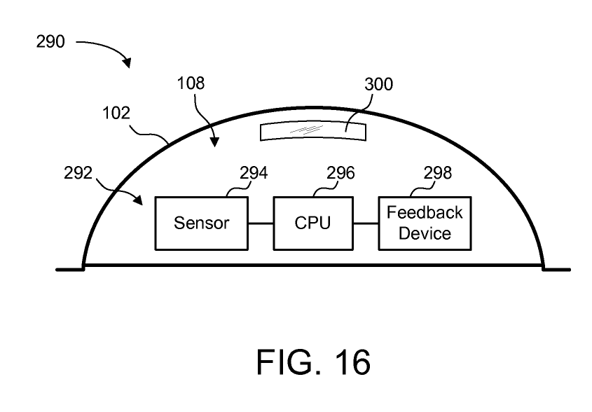

FIG. 16 illustrates a schematic diagram of one embodiment of a NPWT system 290 with a feedback system 292. The illustrated feedback system 292 includes one or more sensors 294, a processing device 296, and a feedback device 298. In general, the feedback system 292 provides operational feedback to a user.

The sensor 294 may include any type of sensor to monitor a variety of operational conditions. In one embodiment, the sensor 294 includes a pressure switch to monitor a vacuum level within the wound interface material 118. In another embodiment, the sensor includes a contact switch to monitor a volume of the fluid within the cavity 108 of the housing 102. For example, the contact switch may be activated as the expandable reservoir 110 expands and makes contact with the contact switch. Other embodiments may incorporate other types of sensors.

The processing device 296 is coupled to the sensor(s) 294. The processing device 296 may include any type of electronic processor (e.g., a central processing unit (CPU)) that is capable of receiving a signal from the sensor(s) 294 and sending an output signal 298 to the feedback device(s) 298. In particular, the processor is configured to generate an activation signal in response to a sensor signal indicative of a general or specific operational condition.

The feedback device 298 may include one or more feedback indicators to provide operational feedback to a user in response. Some examples of feedback devices include, but are not limited to a visual light indicator, an audible speaker, a mechanical vibrator, and a visual display screen. Depending on the type(s) of feedback device(s) implemented in the NPWT system 290, the processing device 296 generates a compatible activation signal, and the feedback device(s) provides corresponding feedback to a user.

In some embodiments, the housing 102 also includes a window 300 to facilitate visual inspection of content within the housing 102. For example, the window 300 may allow a user to visualize the amount of expansion that the expandable reservoir 110 has experienced. In other examples, the window 300 may allow a user to see one or more of the feedback devices 298 within the housing 102.

In the above description, specific details of various embodiments are provided. However, some embodiments may be practiced with less than all of these specific details. In other instances, certain methods, procedures, components, structures, and/or functions are described in no more detail than to enable the various embodiments of the invention, for the sake of brevity and clarity.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

Although specific embodiments of the invention have been described and illustrated, the invention is not to be limited to the specific forms or arrangements of parts so described and illustrated. The scope of the invention is to be defined by the claims appended hereto and their equivalents.

What is claimed is:

1. A wound therapy system comprising:
a wound interface material to contact a wound region and to absorb exudate from the wound region;
a housing to define a cavity in an interior space of the housing;
an osmotic membrane coupled to the wound interface material, the osmotic membrane to transfer a fluid of the exudate from the wound interface material to the cavity of the housing; and
an evacuation port coupled to the wound interface material, the evacuation port to facilitate passage of a gas out of the wound interface material to create a negative pressure at the wound region.

2. The wound therapy system of claim 1, further comprising an expandable reservoir within the cavity of the housing.

3. The wound therapy system of claim 2, wherein the expandable reservoir comprises a bag with at least one elastomeric or other flexible portion.

4. The wound therapy system of claim 2, further comprising an osmogent material within the reservoir, wherein the osmogent is in fluid contact with the osmotic membrane.

5. The wound therapy system of claim 1, wherein the housing comprises at least one material from a plurality of materials, wherein the plurality of materials comprises a rigid material, a semi-rigid material, and a flexible material.

6. The wound therapy system of claim 1, wherein the housing comprises a bottom surface, and the osmotic membrane and the wound interface material are in contact with each other at approximately the bottom surface of the housing.

7. The wound therapy system of claim 6, wherein the bottom surface of the housing comprises a curved surface to accommodate an anatomical feature at the wound region.

8. The wound therapy system of claim 1, wherein the housing comprises a window to facilitate visual inspection of content within the housing.

9. The wound therapy system of claim 1, further comprising:
a dressing attachment coupled to a dressing portion of the wound interface material at the wound region; and
a tube coupled between dressing attachment and the housing, wherein the tube is configured to contain a tube portion of the wound interface material, wherein the tube portion of the wound interface material is configured to transfer the fluid of the exudate from the dressing portion of the wound interface material to the osmotic membrane at the housing, wherein the housing is located remotely from the dressing portion of the wound interface material.

10. The wound therapy system of claim 1, further comprising a sealing member to form a seal around a perimeter of the wound interface material to define a sealed wound environment, wherein the sealing member is impermeable to the gas and the fluid.

11. The wound therapy system of claim 10, wherein the sealing member is in direct physical contact with at least a portion of the wound interface material.

12. The wound therapy system of claim 10, wherein the sealing member is in direct physical contact with a flange on a perimeter of the housing.

13. The wound therapy system of claim 10, wherein the sealing member comprises an occlusive or semi-occlusive film adhesive.

14. The wound therapy system of claim 1, further comprising a mechanical pump coupled to the evacuation port, wherein the pump is configured to be in fluid communication with the wound interface material via the evacuation port, and the pump is further configured to evacuate the gas out of the wound interface material.

15. The wound therapy system of claim 14, wherein the pump is disposed within the housing.

16. The wound therapy system of claim 14, wherein the pump comprises an electromechanical pump.

17. The wound therapy system of claim 14, wherein the pump comprises a manually powered hand pump of an elastomeric material.

18. The wound therapy system of claim 14, wherein the pump comprises a reciprocating pump, a gear-driven pump, a crank-driven pump, a progressing cavity pump, peristaltic pump, or a diaphragm pump.

19. The wound therapy system of claim 14, further comprising a relief port coupled to the wound interface material, the relief port to facilitate passage of an ambient gas into of the wound interface material in response to the negative pressure below a threshold at the wound region.

20. The wound therapy system of claim 19, wherein the relief port is configured to maintain the negative pressure within a range of approximately 25-500 mmHg.

21. The wound therapy system of claim 19, wherein the relief port is configured to maintain the negative pressure at approximately 125 mmHg.

22. The wound therapy system of claim 14, wherein the pump is coupled to an exterior surface of the housing.

23. The wound therapy system of claim 14, wherein the pump is disposed within the internal cavity of the housing.

24. The wound therapy system of claim 14, wherein the pump is detachable from the wound interface material.

25. The wound therapy system of claim 14, wherein the pump is disposed remotely from the wound interface material, and the system further comprises a gas channel between the pump and the evacuation port.

26. The wound therapy system of claim 25, further comprising a three-way valve coupled to the gas channel between the pump and the evacuation port, wherein the three-way valve comprises the evacuation port, a discharge port to discharge the gas evacuated out of the wound interface material into an ambient space, and a relief port to maintain the negative pressure above a threshold.

27. The wound therapy system of claim 26, wherein the three-way valve further comprises a pressure indicator check valve and a feedback window, wherein a position of the pressure indicator check valve relative to the feedback window is indicative of a level of the negative pressure within the wound interface material.

28. The wound therapy system of claim 1, wherein the wound interface material comprises at least one material from a plurality of materials, wherein the plurality of materials comprises a wicking material, an absorptive material, and a hydrophobic material.

29. The wound therapy system of claim 1, wherein the wound interface material comprises an antimicrobial agent and/or a medicinal agent.

30. The wound therapy system of claim 1, wherein the wound interface material comprises one or more layers from a plurality of layers, wherein the plurality of layers comprises a wound contact layer, and exudate transfer layer, an exudate control layer, a cellular material management layer, a wound protein management layer, an exudate solids management layer, a bio-burden control layer, an order management layer, a wound environment monitoring layer, and a growth factor delivery layer.

31. The wound therapy system of claim 1, wherein a top surface of the wound interface material is configured to be flush with a bottom surface of the housing, and a bottom surface of the wound interface material is configured to extend outward from the bottom surface of the housing.

32. The wound therapy system of claim 1, further comprising a filter coupled in fluid communication with the evacuation port, wherein the filter is configured to restrict transfer of fluid and/or solid components of the exudate through the evacuation port.

33. The wound therapy system of claim 32, wherein the filter comprises an antibacterial material to restrict transfer of bacteria from the wound region out of the wound interface material.

34. The wound therapy system of claim 1, further comprising a feedback system to provide operational feedback to a user.

35. The wound therapy system of claim 34, wherein the feedback system comprises:
a sensor to monitor for an operational condition;
a processor coupled to the sensor, wherein the processor is configured to generate an activation signal in response to a sensor signal indicative of the operational condition; and
a feedback indicator coupled to the processor, wherein the feedback indicator is configured to provide the operational feedback to the user in response to the activation signal from the processor.

36. The wound therapy system of claim 35, wherein the sensor comprises a pressure switch to monitor a vacuum level within the wound interface material.

37. The wound therapy system of claim 35, wherein the sensor comprises a contact switch to monitor a volume of the fluid within the cavity of the housing.

38. The wound therapy system of claim 35, wherein the feedback indicator comprises at least one indicator from a plurality of indicators, wherein the plurality of indicators comprises:
a visual light indicator;
an audible speaker;
a mechanical vibrator; and
a visual display screen.

39. A negative pressure wound therapy (NPWT) system comprising:
a wound interface material to contact a wound region and to absorb exudate from the wound region;
an osmotic membrane coupled to the wound interface material, the osmotic membrane to transfer a fluid of the exudate out of the wound interface material; and
a pump coupled to the wound interface material via an evacuation port, wherein the pump is configured to be in fluid communication with the wound interface material and to pump a gas out of the wound interface material to create a negative pressure at the wound region.

40. The wound therapy system of claim 39, further comprising an expandable reservoir coupled to the osmotic membrane, wherein the expandable reservoir is configured to store the fluid transferred out of the wound interface material by the osmotic membrane.

41. The wound therapy system of claim 40, further comprising an osmogent material within the reservoir, wherein the osmogent is in fluid contact with the osmotic membrane.

42. The wound therapy system of claim 40, wherein a top surface of the wound interface material is configured to be flush with a bottom surface of the expandable reservoir.

43. The wound therapy system of claim 39, further comprising a sealing member to form a seal around a perimeter of the wound interface material to define a sealed wound environment, wherein the sealing member is impermeable to the gas and the fluid.

44. The wound therapy system of claim 39, wherein the pump comprises an electromechanical pump.

45. The wound therapy system of claim 39, wherein the pump comprises a manually powered hand pump.

46. The wound therapy system of claim 39, further comprising a relief port coupled to the wound interface material, the relief port to facilitate passage of an ambient gas into of the wound interface material in response to the negative pressure below a threshold at the wound region.

47. The wound therapy system of claim 39, wherein the pump is detachable from the wound interface material.

48. The wound therapy system of claim 39, wherein the pump is disposed remotely from the wound interface material, and the system further comprises a gas channel between the pump and the evacuation port.

49. The wound therapy system of claim 48, further comprising a three-way valve coupled to the gas channel between the pump and the evacuation port, wherein the three-way valve comprises the evacuation port, a discharge port to discharge the gas evacuated out of the wound interface material into an ambient space, and a relief port to maintain the negative pressure above a threshold.

50. The wound therapy system of claim 49, wherein the three-way valve further comprises a pressure indicator check valve and a feedback window, wherein a position of the pressure indicator check valve relative to the feedback window is indicative of a level of the negative pressure within the wound interface material.

51. The wound therapy system of claim 39, further comprising a filter coupled in fluid communication with the pump, wherein the filter is configured to restrict transfer of fluid and/or solid components of the exudate through the pump.

52. A wound therapy system comprising:
a housing to define a cavity in an interior space of the housing;
an osmotic membrane disposed at an opening to the cavity of the housing;
a wound interface material in direct physical contact with at least a portion of the osmotic membrane, wherein the wound interface material is configured to absorb exudate from a wound region, and the osmotic membrane is configured to transfer a fluid of the exudate from the wound interface material to the cavity of the housing; and
a pump coupled to the wound interface material, wherein the pump is configured to at least partially evacuate a gas out of the wound interface material to create a negative pressure at the wound region.

53. The wound therapy system of claim 52, further comprising an osmogent material in contact with the osmotic membrane, on an opposite side from the wound interface material, wherein the osmogent is configured to facilitate osmotic transfer of the fluid from the wound interface material through the osmotic membrane.

54. The wound therapy system of claim 52, further comprising:
a tube coupled to the housing at the opening; and
a dressing attachment coupled to the tube, wherein the wound interface material is couple to the dressing attachment and passes through the dressing attachment and the tube to transfer the fluid of the exudate from the wound region to the osmotic membrane at the opening of the housing.

55. The wound therapy system of claim 52, further comprising a gas channel between the pump and the wound interface material, wherein the pump is disposed remotely from the wound interface material.
